(19) United States
(12) Patent Application Publication
(10) Pub. No.: US 2016/0262807 A1
(43) Pub. Date: Sep. 15, 2016

(54) SURGICAL INSTRUMENT AND METHOD

(71) Applicant: Warsaw Orthopedic, Inc., Warsaw, IN (US)

(72) Inventors: Nicholas M. Benson, Cordova, TN (US); Jason M. May, Cordova, TN (US)

(21) Appl. No.: 14/645,161

(22) Filed: Mar. 11, 2015

(51) Int. Cl. A61B 17/70

(52) U.S. Cl. CPC A61B 17/7076

(57) ABSTRACT

The surgical instrument includes a first implant support being connectable with a fastener. A second implant support is connectable with a fastener. A track is disposed at a selected trajectory to orient translation of at least one of the implant supports along the trajectory. In some embodiments, systems and methods are disclosed

### TECHNICAL HELD

The present disclosure generally relates to medical devices for the treatment of spinal disorders, and more particularly to a surgical instrument and method for correction of a spine disorder.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes discectomy, laminectomy, fusion and implantable prosthetics. Correction treatments used for positioning and alignment of vertebrae may employ implants, such as, for example, spinal constructs and interbody devices, for stabilization of a treated section of a spine. In some embodiments, the spinal constructs may be manipulated with surgical instruments for compression and distraction of vertebrae. This disclosure describes an improvement over these prior art technologies.

### SUMMARY

In one embodiment, a surgical instrument is provided. The surgical instrument includes a first implant support being connectable with a fastener. A second implant support is connectable with a fastener. A track is disposed at a selected trajectory to orient translation of at least one of the implant supports along the trajectory. In some embodiments, systems and methods are disclosed.

### BRIEF DESCRIPTION OF THE DRAWINGS

The present disclosure will become more readily apparent from the specific description accompanied by the following drawings, in which:

FIG. 1 is a perspective view of components of one embodiment of a spinal correction system in accordance with the principles of the present disclosure.

### DETAILED DESCRIPTION

The exemplary embodiments of the system and related methods of use disclosed are discussed in terms of medical devices for the treatment of musculoskeletal disorders and more particularly, in terms of a surgical system and method for correction of a spine disorder.

In one embodiment, the system includes a surgical instrument that can compress or distract and restore curvature of a spine. In one embodiment, the surgical instrument is used to restore vertebral body height and lordosis after a trauma. In one embodiment, the system includes a surgical instrument that can attach to implant supports, such as, for example, screw extenders via clips. In one embodiment, the surgical instrument includes a rack to control movement.

In some embodiments, the surgical instrument allows compression about a selected path, trajectory and/or rotation axis to allow a surgeon to control and predict a sagittal plane correction of vertebrae. In some embodiments, the surgical instrument allows controlled compression and/or lordosis on demand. In some embodiments, the surgical instrument includes a track that guides the implant supports and is adjustable and/or disposable at a selected arcuate path, trajectory and/or rotational axis to provide a surgeon control when performing compression. For example, by selectively controlling a trajectory of the implant support, undesired compression of tissue, such as, for example, nerves roots is resisted and/or prevented during compression maneuvers. In some embodiments, the surgical instrument allows a surgeon to determine the delta and/or angle of change by including visual indicia and/or tactile indicia, such as, for example, an angular calibrated rack on the surgical instrument. In some embodiments, the surgical instrument includes a rack that can placed on or over extenders and/or dips connected with extenders for assembly of the components. In some embodiments, the implant support can include a scissor, forceps and/or joystick.

In some embodiments, one or all of the components of the system may be disposable, peel pack and/or pre packed sterile devices. One or all of the components of the system may be reusable. The system may be configured as a kit with multiple sized and configured components.

In some embodiments, the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In some embodiments, the present disclosure may be employed with other osteal and bone related applications, including those associated with diagnostics and therapeutics. In some embodiments, the disclosed system may be alternatively employed in a surgical treatment with a patient in a prone or supine position, and/or employ various surgical approaches to the spine, including anterior, posterior, posterior mid-line, direct lateral, postero-lateral, and/or antero lateral approaches, and in other body regions. The present disclosure may also be alternatively employed with procedures for treating the lumbar, cervical, thoracic and pelvic regions of a spinal column. The system and methods of the present disclosure may also be used on animals, bone models and other non-living substrates, such as, for example, in training, testing and demonstration.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, in some embodiments, as used in the specification and including the appended claims, the singular forms “a,” “an,” and “the” include the plural, and reference to a particular numerical value includes at least that particular value, unless the context dearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, vessels, ligaments, tendons, cartilage and/or bone unless specifically referred to otherwise.

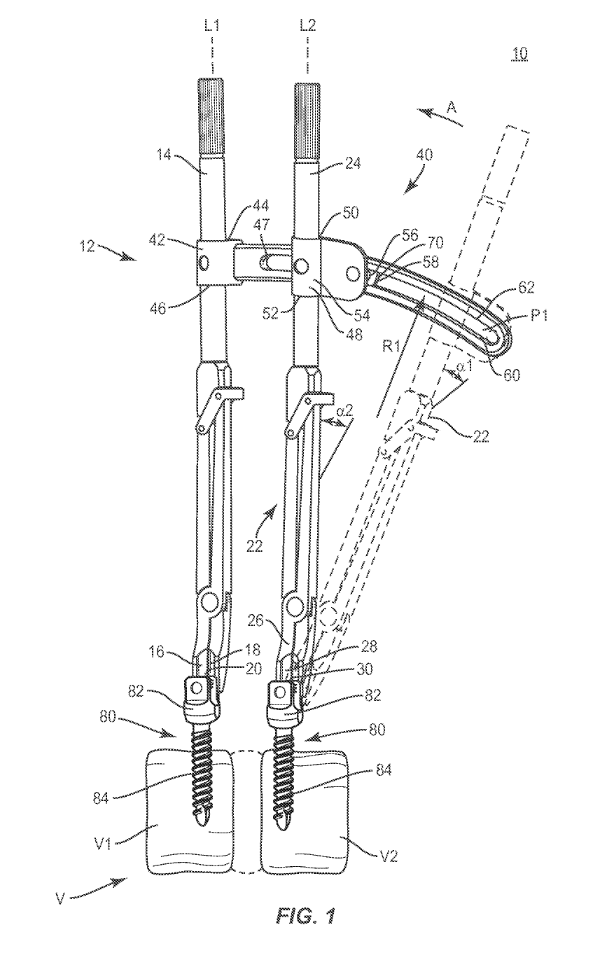

The following discussion includes a description of a system in accordance with the principles of the present disclosure. Alternate embodiments are also disclosed. Reference is made in detail to the exemplary embodiments of the present disclosure, which are illustrated in the accompanying FIGURES. Turning to FIG. 1, there are illustrated components of a system, such as, for example, a spinal correction system 10.

The components of system 10 can be fabricated from biologically acceptable materials suitable for medical applications, including metals, synthetic polymers, ceramics, bone material, tissue and/or their composites, depending on the particular application and/or preference of a medical practitioner. For example, the components of system 10, individually or collectively, can be fabricated from materials such as stainless steel alloys, aluminum, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of system 10 may have material composites, including the above materials, to achieve various desired characteristics such as strength, rigidity, elasticity, compliance, biomechanical performance, durability and radiolucency or imaging preference. The components of system 10, individually or collectively, may also be fabricated from a heterogeneous material such as a combination of two or more of the above-described materials. The components of system 10 may be monolithically formed, integrally connected or include fastening elements and/or instruments, as described herein.

System 10 includes an implant support, such as, for example, an extender 12. Extender 12 extends between an end 14 and an end 16 and defines a longitudinal axis L1. End 14 is configured for connection with a surgical instrument, such as, for example, an instrument 40. End 16 is configured for connection with a fastener, such as, for example, a bone screw 80. End 16 defines an inner surface 18 that defines a cavity 20 configured for disposal of bone screw 80. Surface 18 includes one or more mating elements that engage and removably lock with one or more mating elements of an arm of a receiver of bone screw 80, as described herein. In some embodiments, extender 12 has a variously configured cross section configuration, such as, for example, oval, oblong, triangular, rectangular, square, polygonal, irregular, uniform, non-uniform, variable and/or tapered.

System 10 includes an implant support, such as, for example an extender 22. Extender 22 extends between an end 24 and an end 26 and defines a longitudinal axis L2. End 24 is configured for connection with instrument 40. End 26 is configured for connection with bone screw 80. End 26 defines an inner surface 28 that defines a cavity 30 configured for disposal of bone screw 80. Surface 28 includes one or more mating elements that engage and removably lock with one or more mating elements of an arm of a receiver of bone screw 80, as described herein. In some embodiments, extender 22 has a variously configured cross section configuration, such as, for example, oval, oblong, triangular, rectangular, square, polygonal, irregular, uniform, non-uniform, variable and/or tapered.

In some embodiments, instrument 40 is configured for engagement with extenders 12, 22 to correct a spinal disorder, such as, for example, curvature abnormalities, trauma and/or fracture of vertebrae, which may include a sagittal deformity and requires compression, as described herein. Instrument 40 includes an engagement portion, such as, for example, a clip 42 having an inner surface 44. Surface 44 defines a cavity 46 configured for disposal of a portion of instrument 40, as described herein. Clip 42 mounts to extender 12 and cavity 46 is configured for disposal of instrument 40 to releasably fix instrument 40 with extender 12 at a fixed point on instrument 40 to facilitate translation of extender 22 relative to extender 12, as described herein. In some embodiments, all or only a portion of surface 44 may have alternate surface configurations such as, for example, rough, arcuate, undulating, mesh, porous, semi-porous, dimpled and/or textured. In some embodiments, extender 12 may be integrally connected or monolithically formed with instrument 40 and/or clip 42.

Instrument 40 includes an engagement portion, such as, for example, a clip 48 having an inner surface 50. Surface 50 defines a cavity 52 configured for disposal of a portion of instrument 40, such as, for example, a track 62. Cavity 52 is configured for disposal of track 62 such that dip 48 and extender 22 are movably mounted with instrument 40 and can selectively translate along track 62 to facilitate translation of extender 22 relative to extender 12. In some embodiments, translation of extender 22 relative to extender 12 is caused by distraction and/or compression of vertebrae connected with bone screws 80. Clip 48 includes an outer surface 54. A projection 56 extends from surface 54 and is configured for disposal with track 62 of instrument 40, as discussed herein. Projection 56 extends from surface 54 and includes an end 58 configured for translation along track 62. In some embodiments, extender 22 may be integrally connected or monolithically formed with instrument 40 and/or dip 48.

Instrument 40 includes a surface 47 that defines track 62. In some embodiments, track 62 is integrally connected with dip 42. In some embodiments, track 62 is monolithically formed with dip 42. In some embodiments, track 62 is connected to dip 42 via a hinge (not shown) to allow for lateral movement of track 62 relative to extender 12. In some embodiments, the hinge may be variously configured such as, for example, pin, post, screw, living hinge, ratchet and/or concentric parts.

Instrument 40 includes an inner surface 60 that defines a slot of track 62 that defines, for example, a pathway P1. Track 62 extends along a portion of instrument 40 in an arcuate configuration at a selected trajectory to orient translation of extenders 12, 22. Track 62 defines pathway P1 for translation of projection 56 to translate and/or rotate extender 22. In some embodiments, pathway P1 has various configurations, such as, for example, irregular, uniform, non-uniform, variable and/or tapered. Pathway P1 has an arcuate configuration having a radius of curvature R1 defining an arcuate pathway P1, as shown in FIG. 1.

In one embodiment, surface 60 may include a gear rack (not shown) engageable with projection 56. In one embodiment, track 62 includes visual indicia and/or tactile indicia displaying angular calibration to show a change of an angle α relative to pathway P1. Angle α is defined by relative orientation of extender 22 along pathway P1, for example, between a first position of extender 22 and axis L2 (shown in phantom in FIG. 1) relative to a second position of extender 22 and axis L2.

Instrument 40 includes a lock 70 that is configured to resist translation of extender 22 along track 62. Lock 70 is configured to connect with projection 56 to selectively fix extender 22 relative to extender 12 along pathway P1, for example, in connection with compression and/or distraction of vertebrae.

System 10 includes a fastener, such as, for example, a bone screw 80. Bone screw 80 includes a head 82 configured for attachment with extenders 12, 22 and an elongated shaft 84 configured for penetrating tissue. Shaft 84 has a cylindrical cross section configuration and includes an outer surface having an external thread form. In some embodiments, the thread form may include a single thread turn or a plurality of discrete threads. In some embodiments, other engaging structures may be disposed on shaft 84, such as, for example, a nail configuration, barbs, expanding elements, raised elements and/or spikes to facilitate engagement of shaft 84 with tissue, such as, for example, vertebrae.

In some embodiments, all or only a portion of shaft 84 may have alternate cross section configurations, such as, for example, oval, oblong, triangular, square, polygonal, irregular, uniform, non-uniform, offset, staggered, undulating, arcuate, variable and/or tapered. In some embodiments, the outer surface may include one or a plurality of openings. In some embodiments, all or only a portion of the outer surface may have alternate surface configurations to enhance fixation with tissue such as, for example, rough, arcuate, undulating, mesh, porous, semi-porous, dimpled and/or textured. In some embodiments, all or only a portion of shaft 84 may be disposed at alternate orientations, relative to a longitudinal axis of bone fastener 80, such as, for example, transverse, perpendicular and/or other angular orientations such as acute or obtuse, co-axial and/or may be offset or staggered. In some embodiments, all or only a portion of shaft 84 may be cannulated.

In assembly, operation and use, spinal correction system 10, similar to the systems and methods described herein, is employed with a surgical procedure for treatment of a spinal disorder affecting a section of a spine of a patient, as discussed herein. For example, system 10 can be used with a surgical procedure for treatment of a condition or injury of an affected section of the spine including vertebrae. In some embodiments, one or all of the components of system 10 can be delivered as a pre-assembled device or can be assembled in situ. System 10 may be completely or partially revised, removed or replaced.

For example, system 10 can be employed with a surgical treatment of an applicable condition or injury of an affected section of a spinal column and adjacent areas within a body, such as, for example, vertebrae V. In some embodiments, system 10 may be employed with one or a plurality of vertebra. To treat a selected section of the vertebrae, a medical practitioner obtains access to a surgical site including the vertebrae in any appropriate manner, such as through incision and retraction of tissues. In some embodiments, system 10 can be used in any existing surgical method or technique including open surgery, mini-open surgery, minimally invasive surgery including percutaneous surgical implantation, whereby the vertebrae are accessed through a mini-incision, or sleeve that provides a protected passageway to the area. Once access to the surgical site is obtained, the particular surgical procedure can be performed for treating the spine disorder.

An incision is made in the body of a patient and a cutting instrument (not shown) creates a surgical pathway for delivery of implantable components of system 10 such as, for example, bone screws 80, as shown in FIG. 1. A preparation instrument (not shown) can be employed to prepare tissue surfaces of vertebrae V1, V2, as well as for aspiration and irrigation of a surgical region.

Pilot holes or the like are made in selected vertebra V1 and V2 of vertebrae V for receiving bone screws 80. A driver (not shown) is disposed adjacent vertebrae V at a surgical site and is manipulated to drive, torque, insert or otherwise connect bone screws 80 adjacent vertebrae V1 and V2. Extenders 12, 22 are delivered to the surgical site adjacent vertebrae V and oriented for manipulation, alignment and capture of bone screws 80.

Instrument 40 is engaged with extenders 12, 22 via clips 42, 48, as described herein. Projection 56 is engaged with track 62, as described herein. Axis L2 of extender 22 is disposed at an initial angle α1 (shown in phantom in FIG. 1). In some embodiments, extender 22 is translated and/or rotated along track 62 and/or relative to vertebrae V1, V2 due to compression and/or distraction of V1, V2 due to, for example, interbody implant trialing and/or manipulation of extenders 12, 22 during a surgical procedure.

Extender 22 is translated along pathway P1, as shown by arrow A, such that projection 56 translates along pathway P1 to rotate and/or translate extender 22 relative to extender 12 to compress vertebrae V1, V1. As extender 22 rotates, angle α changes to compress vertebrae V1, V2 to a second position at angle α2. Extender 22 is translatable and/or rotatable to the second position and can be locked positionally and/or incrementally via lock 70, which may comprise a ratchet and/or torsion lock.

Upon completion of a procedure, surgical instrument 40, surgical instruments and/or tools, assemblies and non-implanted components of system 10 are removed and the incision(s) are dosed. One or more of the components of system 10 can be made of radiolucent materials such as polymers. Radiomarkers may be included for identification under x-ray, fluoroscopy, CT or other imaging techniques. In some embodiments, the use of surgical navigation, microsurgical and image guided technologies may be employed to access, view and repair spinal deterioration or damage, with the aid of system 10. In some embodiments, system 10 may include one or a plurality of plates, connectors and/or bone fasteners for use with a single vertebral level or a plurality of vertebral levels.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A surgical instrument comprising:
a first implant support being connectable with a fastener;
a second implant support being connectable with a fastener; and
a track disposed at a selected trajectory to orient translation of at least one of the implant supports along the trajectory.

2. A surgical instrument as recited in claim 1, wherein the track includes an inner surface that defines a slot configured for translation of the second implant support to facilitate compression.

3. A surgical instrument as recited in claim 1, wherein the track includes an inner surface that defines a cavity configured for disposal of the second implant support for translation of the second implant support relative to the first implant support.

4. A surgical instrument as recited in claim 1, wherein the first implant support is fixedly connected with the track to define a rotational axis point along a sagittal plane.

5. A surgical instrument as recited in claim 1, further comprising a clip that mounts the first implant support with the track.

6. A surgical instrument as recited in claim 1, wherein the first implant support includes a cavity configured to receive a fastener and the second implant support includes a cavity configured to receive a fastener.

7. A surgical instrument as recited claim 1, wherein the track includes an arcuate slot.

8. A surgical instrument as recited in claim 1, wherein the track includes indicia displaying an angle of rotation.

9. A surgical instrument as recited in claim 1, wherein the first implant support includes an extender.

10. A surgical instrument as recited in claim 1, wherein the second implant support includes an extender.

11. A surgical instrument as recited in claim 1, wherein the track includes a lock configured to fix the second implant support in a position relative to the first implant support.
